(19) United States
(12) Patent Application Publication
(10) Pub. No.: US 2011/0230450 A1
(43) Pub. Date: Sep. 22, 2011

(54) PLATELET-ACTIVATING FACTOR (PAF) ANALOGS AND USES THEREOF

(75) Inventors: Itzhak Mendel, Rechovot (IL); Niva Yacov, Tel-Aviv (IL); Eyal Breitbart, Hashmonaim (IL)

(73) Assignee: Vascular Biogenics Lts, Or Yehuda (IL)

(21) Appl. No.: 12/738,097

(22) PCT Filed: Oct. 5, 2008
(86) PCT No.: PCT/IL2008/001315

§ 371 Date: Apr. 15, 2010

Related U.S. Application Data
(62) Division of application No. 60/960,846, filed on Oct. 16, 2007.

(51) Int. Cl. A61K 31/685; A61P 29/00

(52) U.S. Cl. 514/129

(57) ABSTRACT

Disclosed are novel methods and compositions for treating inflammatory diseases and disorders, utilizing structural analogs of platelet activating factor (PAF).

### FIELD AND BACKGROUND OF THE INVENTION

The present invention, in some embodiments thereof, relates to novel methods for treating or preventing an inflammation, and more particularly, but not exclusively, to methods and compositions which utilize structural analogs of PAF (platelet-activating factor).

PAF is a phospholipid having a general structure of a 1-alkyl-2-acetyl-sn-glycero-3-phosphocholine, wherein the alkyl group is typically hexadecyl, although PAF species containing other alkyl groups, such as octadecyl, are known. The structure of PAF thus differs from that of typical phospholipids in that (i) typical phospholipids have two acyl groups at positions sn-1 and sn-2 rather than an alkyl group at sn-1 and an acyl group at sn-2, and (ii) the acyl groups in typical phospholipids are considerably longer than the acetyl group in PAF. The structures of PAF and typical phospholipids are shown in Scheme 1 below.

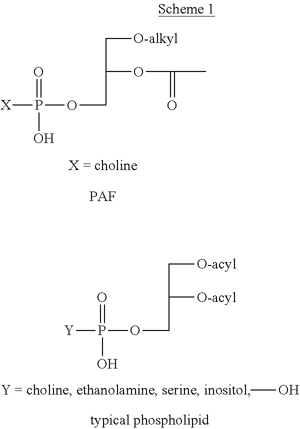

PAF is produced by cells such as neutrophils, basophils, platelets and endothelial cells, and is a potent mediator of various biological functions, such as platelet aggregation, inflammation and anaphylaxis.

Several reports have demonstrated that oxidation of LDL (low-density lipoprotein) could result in the generation of PAF-like lipids [Tokumura et al., 1996; Heery et al., 1995; Marathe et al., 1999]. The binding of some PAF-like lipids to the PAF receptor was reported to increase adhesion and calcium flux of monocytes and neutrophils, suggesting a pro-inflammatory role for PAF analogs [Lehr et al., 1997; Smiley et al., 1991; Marathe et al., 2002], consistent with the role of PAF as a mediator of inflammation.

Other reports have suggested an anti-inflammatory role for molecules derived from LDL oxidation [Bluml et al., 2005; Walton et al., 2003; Bochkov et al., 2002]. However, such molecules generally include acyl groups, as is typical of lipids, rather than alkyl groups, as well as oxidized moieties such as aldehyde and carboxylic acid moieties [Walton et al., 2003].

International Patent Application WO 06/086992 discloses lysophosphatidic acid analogs such as monoether lysolipids, as well as ester-linked lipid derivatives thereof which may be hydrolyzed by PLA₂, thereby serving as prodrugs of the lysophosphatidic acid analog. Pharmaceutical compositions containing the lipid derivatives are described therein as being suitable for treating cancer, infectious and inflammatory conditions.

U.S. Pat. No. 5,352,810 discloses phosphatidylinositol analogs, optionally comprising an alkyl group at the sn-1 position and a short chain acyl group (particularly acetyl) at the sn-2 position, for inhibiting phosphatidylinositol-specific phospholipase C, which could be used as anti-tumoral, platelet anti-aggregating and anti-inflammatory drugs.

International Patent Application WO 02/038575 discloses phosphatidylinositol compounds and related compounds, for inhibiting phospholipase A2, and for the treatment of pathologies mediated by phospholipase A2 activity.

U.S. Pat. No. 5,762,958 discloses a liposome comprising an ether lipid analog of phosphatidylcholine, optionally comprising an acyl group at the sn-2 position, and methods of treating cancer or an inflammatory disorder by administration of the liposomes.

Additional related art includes Deigner and Dresel [FEBS Letters (1993), 317:202-206], Lamotte-Brasseur et al. [Biochimica et Biophysica Acta, Lipids and Lipid Metabolism (1991), 1085:91-105] and German Patent No. DE 3212387.

### SUMMARY OF THE INVENTION

The prior art describes structural analogs of PAF mainly as pro-inflammatory agents. The prior art further teaches oxidized phospholipids, lysophosphatidic acid, phosphatidylinositol, and structural analogs thereof as anti-inflammatory agents. The prior art, however, fails to teach or suggest a role for structural analogs of PAF as anti-inflammatory agents.

The present inventors have now surprisingly uncovered that structural analogs of PAF have effective anti-inflammatory properties, and therefore can be utilized as therapeutically effective agents for treating or preventing inflammatory diseases or disorders, such as, for example, multiple sclerosis, arthritis, inflammatory bowel disease (IBD), atherosclerosis and psoriasis.

According to an aspect of some embodiments of the present invention there is provided a method of treating an inflammatory disease or disorder in a subject in need thereof, the method comprising administering the subject a therapeutically effective amount of at least one PAF-analog of general Formula I:

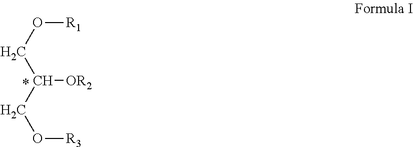

wherein:

* denotes a chiral or non-chiral carbon atom, having a S-configuration and/or a R-configuration;

R₁ is an alkyl chain 16 to 18 carbon atoms in length;

R₂ is selected from the group consisting of hydrogen and an acyl group having the formula:

—C(═O)—(CH₂)n-H

wherein n equals 0, 2, 3, 4, 5, 6 or 7; and

R₃ is selected from the group consisting of phosphate, phosphocholine, phosphoethanolamine, phosphoserine, phosphoinositol, alkyl, aryl, cycloalkyl, carboxy, saccharide, ethylphosphocholine, phosphorylmethanol, phosphorylethanol, phosphorylpropanol, phosphorylbutanol, phosphoethanolamine-N-lactose, phosphoethanolamine-N-[methoxy(propylene glycol)], phosphoinositol-4-phosphate, phosphoinositol-4,5-biphosphonate, pyrophosphate, phosphoethanolamine-diethylenetriamine-pentaacetate, dinitrophenyl-phosphoethanolamine and phosphoglycerol,

or a pharmaceutically acceptable salt thereof,

thereby treating the inflammatory disease or disorder.

According to an aspect of some embodiments of the present invention there is provided a use of a PAF-analog of general Formula I described hereinabove, or a pharmaceutically acceptable salt thereof, in the manufacture of a medicament for treating an inflammatory disease or disorder.

According to an aspect of some embodiments of the present invention there is provided a pharmaceutical composition comprising a PAF-analog of general Formula I described hereinabove, or a pharmaceutically acceptable salt thereof, and a pharmaceutical acceptable carrier, the composition being packaged in a packaging material and identified in print, in or on the packaging material for use in the treatment of an inflammatory disease or disorder.

According to some embodiments of the invention, R₃ is phosphocholine.

According to some embodiments of the invention, R₁ is hexadecyl.

According to some embodiments of the invention, R₂ is hydrogen.

According to some embodiments of the invention, n equals 3.

According to some embodiments of the invention, the PAF-analog is selected from the group consisting of 1-hexadecyl-2-butyroyl-sn-glycero-3-phosphocholine, 1-octadecyl-2-butyroyl-sn-glycero-3-phosphocholine, 1-hexadecyl-2-hydroxy-sn-glycero-3-phosphocholine and 1-octadecyl-2-hydroxy-sn-glycero-3-phosphocholine.

According to some embodiments of the invention, the inflammatory disease or disorder is selected from the group consisting of an idiopathic inflammatory disease or disorder, a chronic inflammatory disease or disorder, an acute inflammatory disease or disorder, an autoimmune disease or disorder, an infectious disease or disorder, an inflammatory malignant disease or disorder, an inflammatory transplantation-related disease or disorder, an inflammatory degenerative disease or disorder, a disease or disorder associated with a hypersensitivity, an inflammatory cardiovascular disease or disorder, an inflammatory cerebrovascular disease or disorder, a peripheral vascular disease or disorder, an inflammatory glandular disease or disorder, an inflammatory gastrointestinal disease or disorder, an inflammatory cutaneous disease or disorder, an inflammatory hepatic disease or disorder, an inflammatory neurological disease or disorder, an inflammatory musculo-skeletal disease or disorder, an inflammatory renal disease or disorder, an inflammatory reproductive disease or disorder, an inflammatory systemic disease or disorder, an inflammatory connective tissue disease or disorder, an inflammatory tumor, necrosis, an inflammatory implant-related disease or disorder, an inflammatory aging process, an immunodeficiency disease or disorder and an inflammatory pulmonary disease or disorder.

According to some embodiments of the invention, the inflammatory disease or disorder is an autoimmune disease or disorder.

According to some embodiments of the invention, the autoimmune disease or disorder is selected from the group consisting of chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

According to some embodiments of the invention, the autoimmune disease or disorder is selected from the group consisting of inflammatory bowel disease, atherosclerosis, psoriasis, chronic rheumatoid arthritis, juvenile rheumatoid arthritis and multiple sclerosis.

According to some embodiments of the invention, the method further comprises administering to the subject a therapeutically effective amount of at least one additional compound capable of treating or preventing the inflammatory disease or disorder.

According to some embodiments of the invention, the medicament is used in combination with a therapeutically effective amount of at least one additional compound capable of treating or preventing the inflammatory disease or disorder.

According to some embodiments of the invention, the pharmaceutical composition further comprises a therapeutically effective amount of at least one additional compound capable of treating or preventing the inflammatory disease or disorder.

According to some embodiments of the invention, the at least one additional compound is selected from the group consisting of a HMGCoA reductase inhibitor (a statin), a mucosal adjuvant, a corticosteroid, a steroidal anti-inflammatory drug, a non-steroidal anti-inflammatory drug, an analgesic, a growth factor, a toxin, a HSP, a beta-2-glycoprotein I, a cholesteryl ester transfer protein (CETP) inhibitor, a peroxisome proliferative activated receptor (PPAR) agonist, an anti-atherosclerosis drug, an anti-proliferative agent, ezetimide, nicotinic acid, an ApoE Milano, and any derivative and analog thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents the molecular structure of PAF (platelet-activating factor; 1-hexadecyl-2-acetoyl-sn-glycero-3-phosphocholine), and of exemplary PAF analogs according to embodiments of the invention: CI-302 (1-hexadecyl-2-butyroyl-sn-glycero-3-phosphocholine) and CI-303 (lyso-PAF; 1-hexadecyl-2-hydroxy-sn-glycero-3-phosphocholine);

FIG. 2 presents comparative plots showing human platelet aggregation over time as indicated by light transmittance (y-axis), following treatment with 0.125 μM (red line), 0.25 μM (blue line) and 0.5 μM (brown line) CI-302 and 0.125 μM PAF (green line);

FIG. 3 presents comparative plots showing human platelet aggregation over time as indicated by light transmittance (y-axis), following treatment with 1 μM (blue line) and 10 μM (brown line) CI-302, 0.25 μM PAF (red line), and phosphate buffer saline as a control (green line);

FIG. 4 presents comparative plots showing human platelet aggregation over time as indicated by light transmittance (y-axis), following treatment with 100 μM (blue line) and 1,000 μM (brown line) CI-302, 0.25 μM PAF (red line), and phosphate buffer saline as a control (green line);

FIGS. 5A-B are bar graphs presenting IL-12/23 p40 expression in bone marrow-derived dendritic cells (BMDCs) activated with 10 μg/ml peptidoglycan, following incubation for 1 hour with various concentrations of CI-302 (FIG. 5A) or CI-303 (FIG. 5B) (each bar represents 4 samples in FIG. 5A, 6 samples in FIG. 5B);

FIG. 6 is a bar graph presenting IL-12/23 p40 expression in CD11c+ bone marrow-derived dendritic cells (BMDCs) 2, 3 and 4 hours after activation with 10 μg/ml peptidoglycan, following incubation for 1 hour with 20 μg/ml CI-302 (hatched column), CI-303 (bricked column) or phosphatidylcholine (PC) (white column), or no treatment (black column); results were normalized according to GAPDH (glyceraldehyde 3-phosphate dehydrogenase) expression, and the p40 expression in non-activated cells was defined as 1;

FIG. 7 presents comparative plots showing the mean clinical score for MOG peptide-induced experimental autoimmune encephalomyelitis (EAE) as a function of time in mice fed 200 μl PBS or 0.04 or 0.4 mg/kg CI-302 in 200 μl PBS daily from 5 days before to 5 days after induction of EAE by immunization of the mice with MOG peptide 35-55 in complete Freund's adjuvant;

FIGS. 8A-F are bar graphs presenting expression of IL-12/23 p40 (FIG. 8A), p35 (FIG. 8B), CCL2 (FIG. 8C), IFN-γ (FIG. 8D), TNF-α (FIG. 8E) and CD4 (FIG. 8F) in spinal cords of mice fed PBS or 0.4 mg/kg CI-302 in PBS daily from 5 days before to 5 days after induction of EAE by immunization of the mice with MOG peptide 35-55 in complete Freund's adjuvant (samples were taken 28 days after immunization), and in spinal cords of naive control mice; results were normalized according GAPDH (glyceraldehyde 3-phosphate dehydrogenase) expression, and mean results in naive mice were defined as 1;

FIG. 9 presents comparative plots showing the mean score of collagen-induced arthritis over time in mice following injections of collagen-II (on days 0 and 21); 0.4 mg/kg CI-302 was administered daily from day 22 to day 39, 0.04 mg/kg CI-201 and methotrexate (MTX) were administered as a positive control, and PBS was administered as a negative control; and

FIG. 10 presents images of a Western blot of phosphotyrosine and ERK1/2 in peritoneal macrophages treated with solvent (1% ethanol/PBS) or 20 μg/ml of phosphatidylcholine, CI-201, or CI-302, (ERK1/2 levels are shown as a loading control).

### DESCRIPTION OF SPECIFIC EMBODIMENTS OF THE INVENTION

The present invention, in some embodiments thereof, relates to novel methods for treating or preventing an inflammation, and more particularly, but not exclusively, to methods and compositions which utilize structural analogs of PAF (platelet-activating factor).

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The prior art teaches that PAF is a potent mediator of inflammation. Indeed, administration of PAF may result in extreme, life-threatening inflammation.

The prior art further suggests that compounds having an analogous structure to that of PAF have pro-inflammatory effects, although weaker than that of PAF.

While reducing the present invention to practice, the present inventors have surprisingly uncovered that administration of structural analogs of PAF can inhibit pro-inflammatory cellular mechanisms and thus inhibit inflammation, in both in vitro and in vivo models.

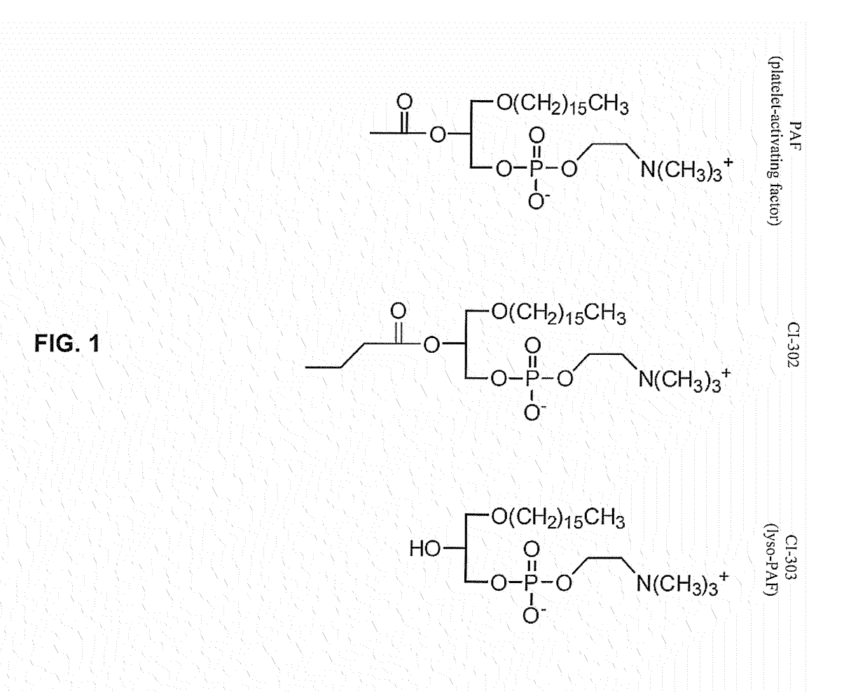

Referring now to the drawings, FIG. 1 illustrates the molecular structure of PAF, as well as those of exemplary the PAF-analogs CI-302 (1-hexadecyl-2-butyroyl-sn-glycero-3-phosphocholine) and CI-303 (lyso-PAF; 1-hexadecyl-2-hydroxy-sn-glycero-3-phosphocholine).

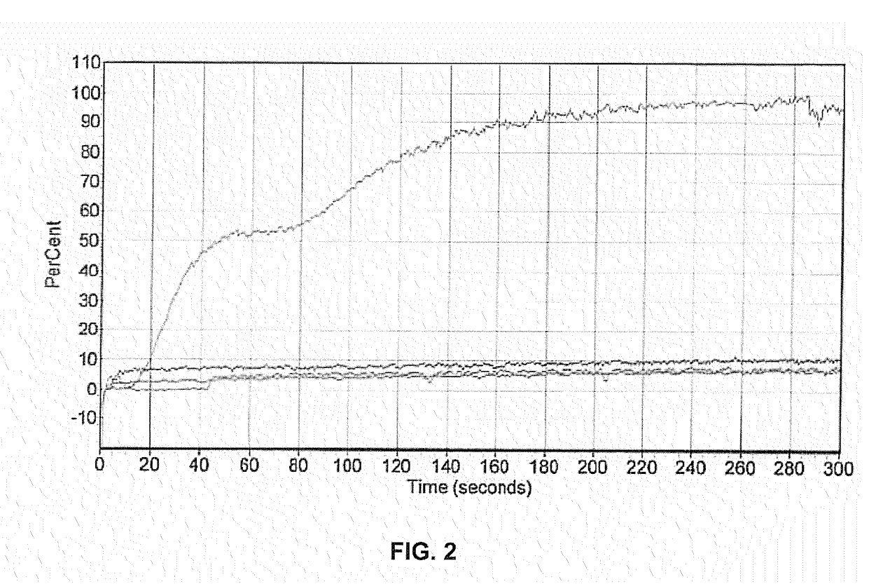

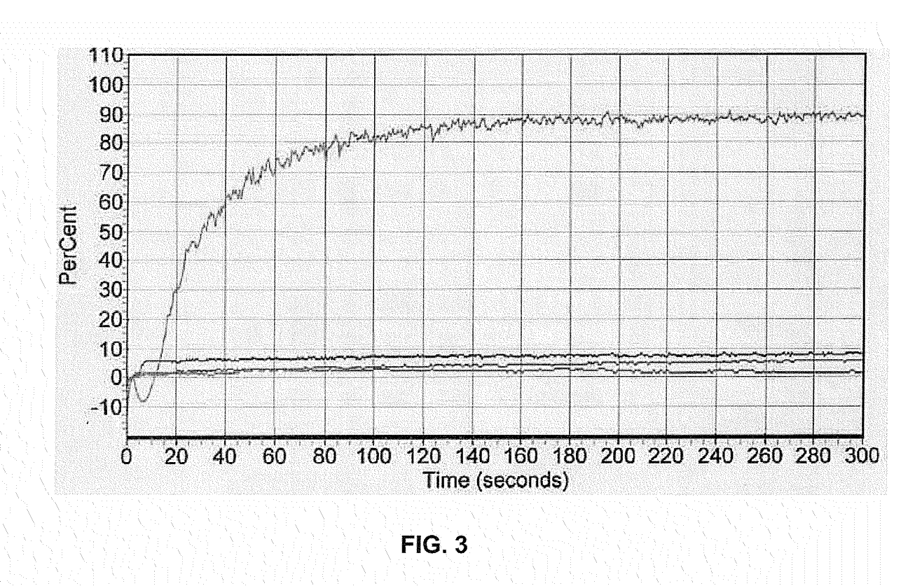

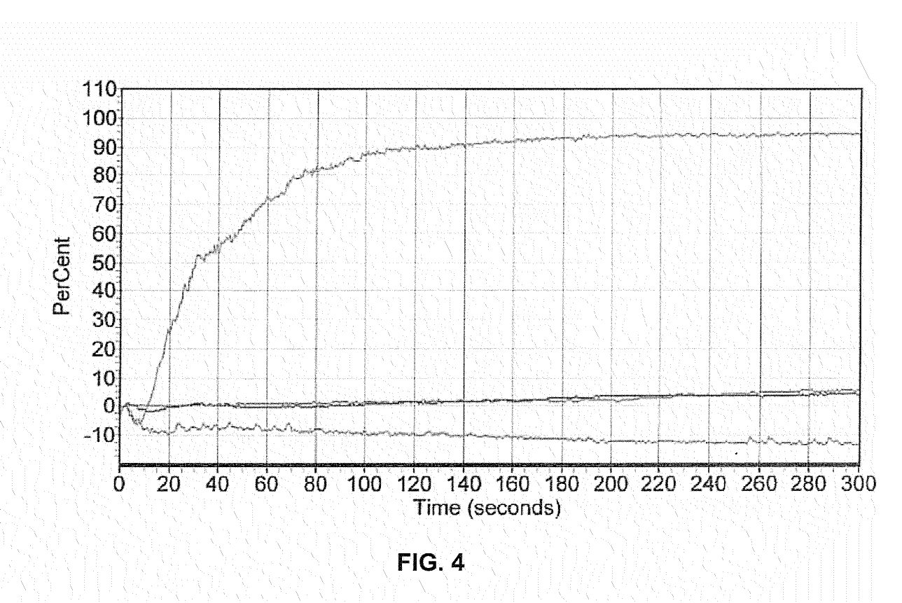

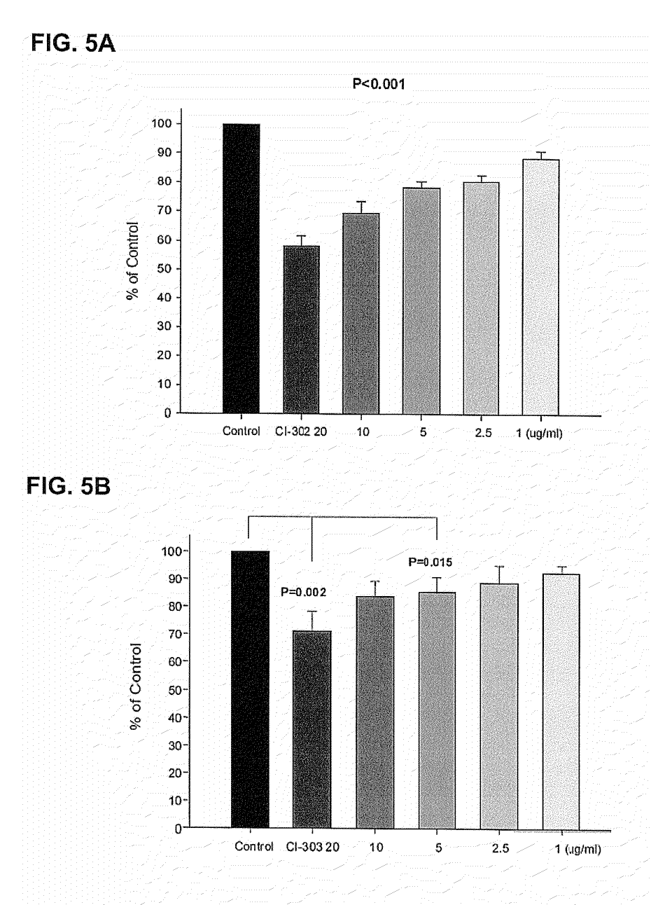

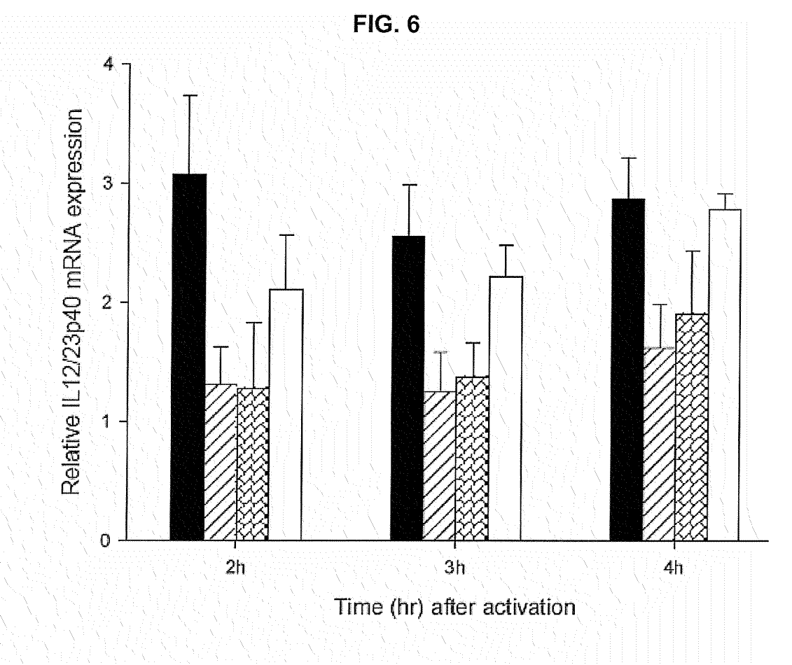

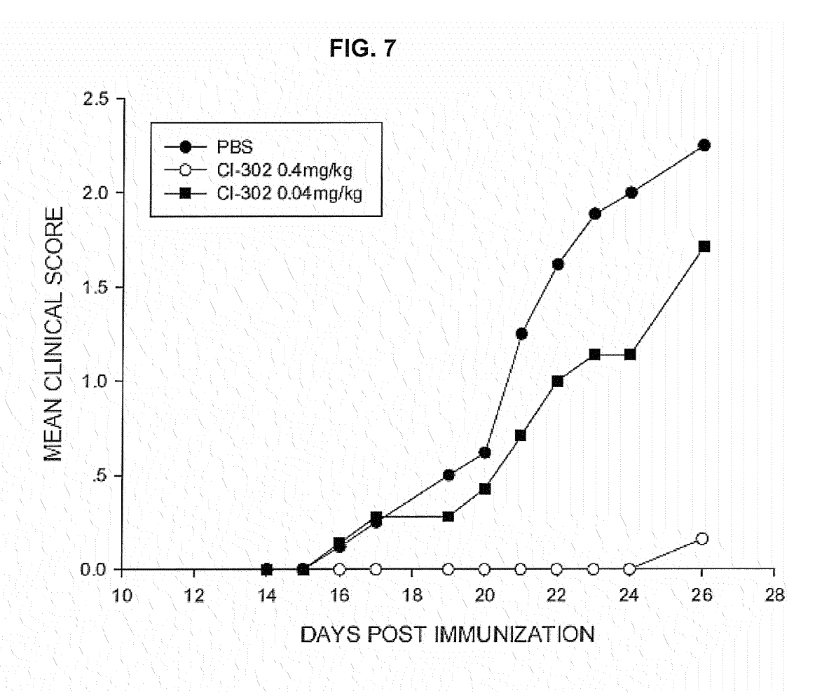

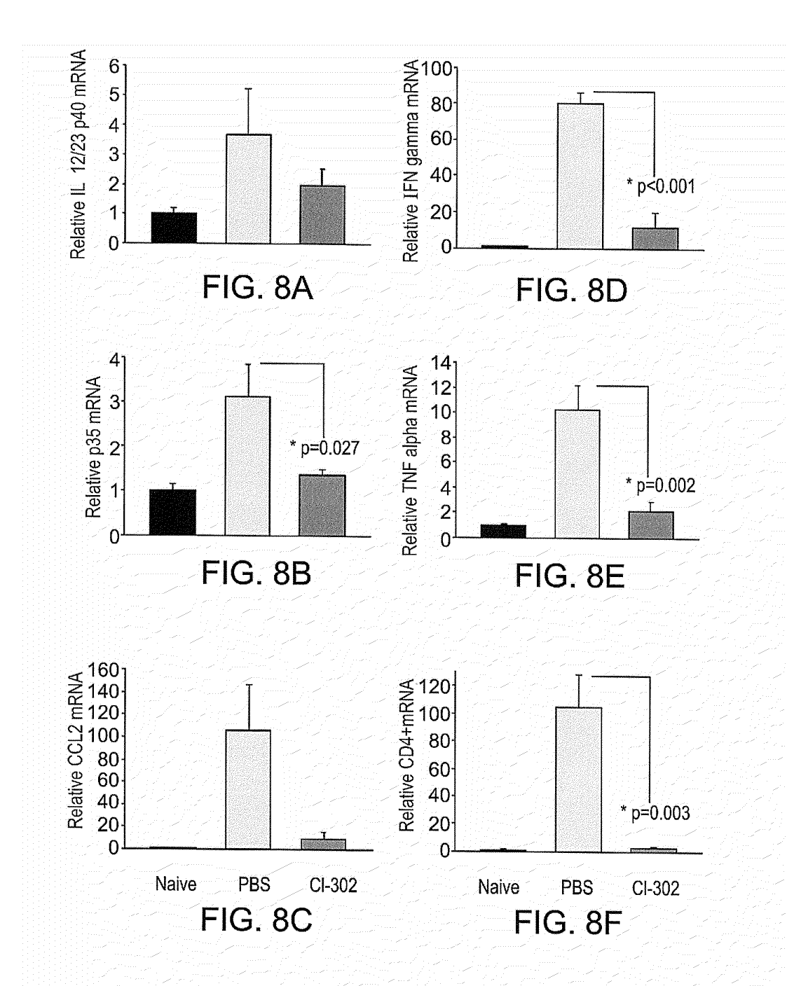

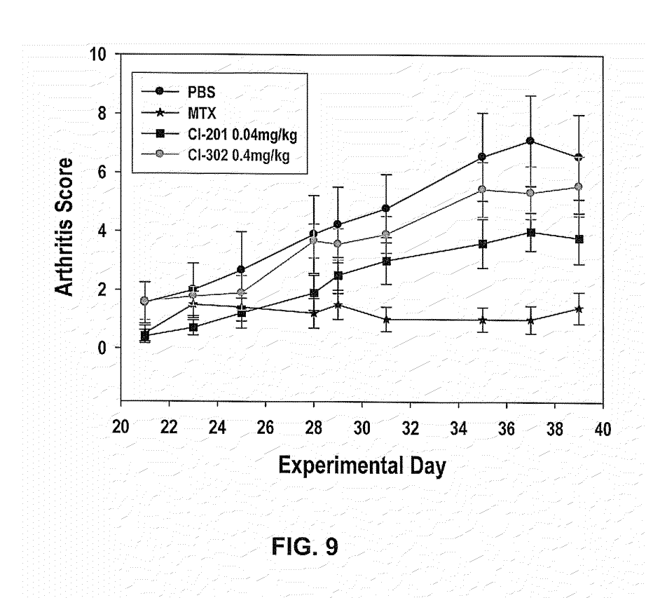

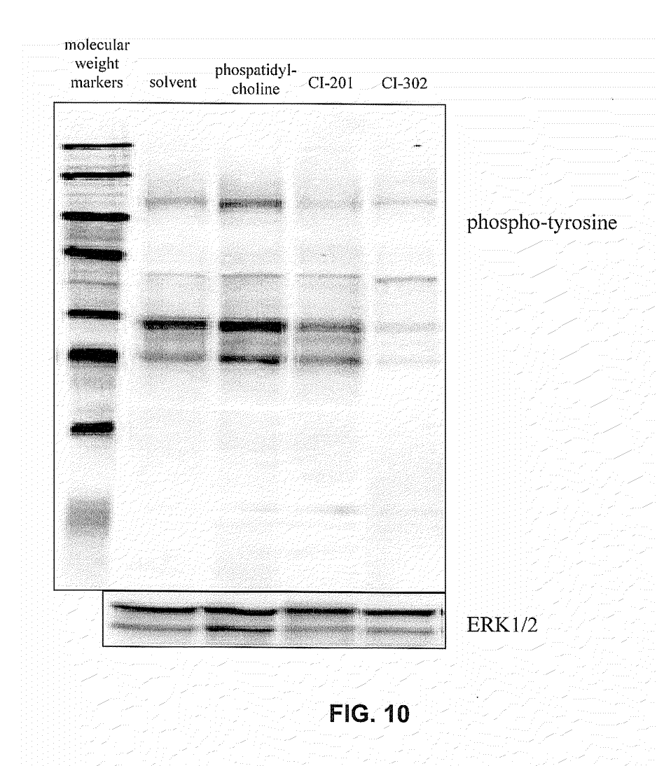

As demonstrated in FIGS. 2-4, the PAF-analog CI-302 lacks the capability to induce platelet aggregation that is characteristic of PAF. However, as demonstrated in FIGS. 5 and 6, both CI-302 and 303 PAF-analogs are effective at inhibiting expression of the pro-inflammatory p40 protein (a component of IL-12 and IL-23). In addition, CI-302 was found to inhibit inflammation in experimental autoimmune encephalomyelitis, an experimental multiple sclerosis model (FIGS. 7 and 8), and in an experimental arthritis model (FIG. 9). Further evidence for the anti-inflammatory activity of PAF-analogs is presented in FIG. 10, wherein the inhibition by CI-302 of tyrosine phosphorylation in macrophages is shown.

Experimental autoimmune encephalomyelitis (EAE) is a CD4+ T-cell mediated inflammatory disease of the central nervous system (CNS) widely used as the animal model for multiple sclerosis (MS). Activation of T helper (Tₕ) cells with their cognate antigens presented by CNS antigen-presenting cells (APCs) is essential for disease initiation [Slavin et al., 2001]. Moreover, activation of infiltrating naïve T-cells specific for novel myelin antigens by CNS APCs promotes remission and relapse episodes during disease course [McMahon et al., 2005]. Largely, microglia and astroglial cells were believed to be the sole APCs in the CNS to induce an effector function by encephalitogenic T-cells [Becher et al., 2000; Fierz et al., 1985; Ford et al., 1996]. However, recent studies have indicated that CD11c+ dendritic cells (DC) are present in the CNS during homeostasis and are sufficient to present antigen to autoreactive T cells [Greter et al., 2005]. Bone-marrow chimera experiments have revealed that DCs found in the CNS at peak disease originated from the periphery and were predominantly of the myeloid DC (mDC) subset [Greter et al., 2005; Deshpande et al., 2007]. Upon activation, CNS mDCs expressed IL-12 and IL-23, and consequently promoted naïve myelin-specific CD4+ T-cell differentiation into Tₕ1 and Tₕ17 cells, respectively [Deshpande et al., 2007; Bailey et al., 2007]. Hence, inhibition of IL-12/23 p40, as demonstrated in the Examples, results in an inhibition of the inflammatory autoimmune reaction.

The obtained data strongly suggest that the PAF analogs presented herein can be beneficially used in treating inflammatory diseases and disorder.

The PAF analogs described herein have a lipid structure (as PAF is a phospholipid), and thus are either natural compounds or structural analogs thereof, and are therefore relatively safe and non-toxic.

Thus, according to one aspect of the present invention there is provided a method of treating an inflammatory disease or disorder in a subject in need thereof, the method comprising administering the subject a therapeutically effective amount of at least one structural analog of PAF, having general Formula I:

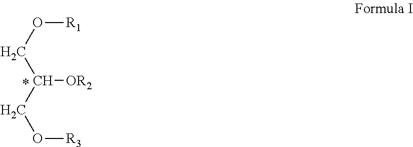

wherein:

* denotes a chiral or non-chiral carbon atom, having a S-configuration and/or a R-configuration;

R₁ is an alkyl chain 16 to 18 carbon atoms in length;

R₂ is selected from the group consisting of hydrogen and an acyl group having the formula:

—C(═O)—(CH₂)n-H

wherein n equals 0, 2, 3, 4, 5, 6 or 7; and

R₃ is selected from the group consisting of phosphate, phosphocholine, phosphoethanolamine, phosphoserine, phosphoinositol, alkyl, aryl, cycloalkyl, carboxy, saccharide, ethylphosphocholine, phosphorylmethanol, phosphorylethanol, phosphorylpropanol, phosphorylbutanol, phosphoethanolamine-N-lactose, phosphoethanolamine-N-[methoxy(propylene glycol)], phosphoinositol-4-phosphate, phosphoinositol-4,5-biposphonate, pyrophosphate, phosphoethanolamine-diethylenetriamine-pentaacetate, dinitrophenyl-phosphoethanolamine and phosphoglycerol,

thereby treating or preventing the inflammatory disease or disorder.

According to some embodiments of the present invention, R₃ is phosphocholine or a chemically related group, such as phosphoethanolamine, phosphoserine, ethylphosphocholine, phosphoethanolamine-N-lactose, phosphoethanolamine-N-[methoxy(propylene glycol)], phosphoethanolamine-diethylenetriamine-pentaacetate or dinitrophenyl-phosphoethanolamine. In some embodiments, R₃ is phosphocholine, phosphoserine or phosphoethanolamine. In some embodiments, R₃ is phosphocholine.

As discussed hereinabove, naturally occurring PAFs have a phosphocholine moiety at the sn-3 position thereof.

According to some embodiments of the present invention, R₁ is hexadecyl or octadecyl (an alkyl chain 16 or 18 carbon atoms in length). In some embodiments, R₁ is hexadecyl.

As discussed hereinabove, naturally occurring PAFs have hexadecyl at position sn-1 thereof, although some have octadecyl.

According to embodiments of the invention, the structural analogs of PAF described herein have at position sn-2 thereof a chemical moiety other than the acyl group present in naturally occurring PAFs.

Thus, in some embodiments, R₂ is hydrogen, in which case the PAF-analog has a structure of a glycerolipid (e.g., PAF) which is hydrolysed at the sn-2 position of the glycerol moiety thereof.

In some embodiments, R₂ is an acyl group (e.g., propanoyl, butyroyl, pentanoyl, hexanoyl). Butyroyl (in which n equals 3 according to the above formula for an acyl group) is an exemplary acyl group.

Exemplary PAF-analogs according to embodiments of the invention include, but are not limited to, 1-hexadecyl-2-butyroyl-sn-glycero-3-phosphocholine, 1-octadecyl-2-butyroyl-sn-glycero-3-phosphocholine, 1-hexadecyl-2-hydroxy-sn-glycero-3-phosphocholine and 1-octadecyl-2-hydroxy-sn-glycero-3-phosphocholine. In the aforementioned compounds, R₁ is either hexadecyl or octadecyl, R₂ is either hydrogen or butyroyl, and R₃ is phosphocholine. All stereoisomers of the abovementioned compounds, as well as mixtures thereof, are included within these embodiments of the invention.

As described herein, the carbon atom at the sn-2 position (marked by *) of the glycerol moiety of the compound of general Formula I is chiral (except for embodiments in which R₁ is identical to R₃). Additionally, any carbon atoms in R₁, R₂ and R₃ can be chiral or non-chiral. Any chiral carbon atom that is present in the compounds of general Formula I, such as the carbon atom at the sn-2 position of the glycerol moiety, can be either in an R-configuration, an S-configuration or a mixture thereof (e.g., a racemic compound). Thus, embodiments of the present invention encompass any combination of chiral and racemic carbon atoms, including all the possible stereoisomers, optical isomers, enantiomers, and anomers.

The embodiments of the present invention further encompass any pharmaceutically acceptable salts, prodrugs, hydrates and solvates of the compounds described hereinabove.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

The term “solvate” refers to a complex of variable stoichiometry (e.g., di-, tri-, tetra-, penta-, hexa-, and so on), which is formed by a solute (the compound of present invention) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

As used herein, the phrase “inflammatory disease or disorder” refers to any disease or disorder associated with an inflammation.

Inflammation is a protective response of the body to an injury. Several cytokines play key roles in mediating inflammatory reactions amongst which are IL-12 (a heterodimer consisting of p40 and p35 proteins), CCL2 (chemokine (C—C motif) ligand 2), IFN-γ (interferon-γ) and TNF-α (tumor necrosis factor α).

Excessive inflammation is oftentimes deleterious, involving or leading to a myriad of diseases and disorders.

As is shown in the Examples section that follows, administration of PAF-analogs is associated with an anti-inflammatory effect. This anti-inflammatory effect may be utilized in treating or preventing inflammatory diseases and/or disorders.

Thus, representative examples of diseases or disorders associated with an inflammation, treatable by the method of the present invention include, but are not limited to, idiopathic inflammatory diseases or disorders, chronic inflammatory diseases or disorders, acute inflammatory diseases or disorders, autoimmune diseases or disorders, infectious diseases or disorders, inflammatory malignant diseases or disorders, inflammatory transplantation-related diseases or disorders, inflammatory degenerative diseases or disorders, diseases or disorders associated with a hypersensitivity, inflammatory cardiovascular diseases or disorders, inflammatory cerebrovascular diseases or disorders, inflammatory peripheral vascular diseases or disorders, inflammatory glandular diseases or disorders, inflammatory gastrointestinal diseases or disorders, inflammatory cutaneous diseases or disorders, inflammatory hepatic diseases or disorders, inflammatory neurological diseases or disorders, inflammatory musculo-skeletal diseases or disorders, inflammatory renal diseases or disorders, inflammatory reproductive diseases or disorders, inflammatory systemic diseases or disorders, inflammatory connective tissue diseases or disorders, inflammatory tumors, necrosis, inflammatory implant-related diseases or disorders, inflammatory aging processes, immunodeficiency diseases or disorders, proliferative diseases and disorders and inflammatory pulmonary diseases or disorders, as are detailed hereinbelow.

Non-limiting examples of hypersensitivities include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Non-limiting examples of inflammatory cardiovascular disease or disorder include occlusive diseases or disorders, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener's granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, and anti-helper T lymphocyte autoimmunity.

Stenosis is an occlusive disease of the vasculature, commonly caused by atheromatous plaque and enhanced platelet activity, most critically affecting the coronary vasculature.

Restenosis is the progressive re-occlusion often following reduction of occlusions in stenotic vasculature. In cases where patency of the vasculature requires the mechanical support of a stent, in-stent-stenosis may occur, re-occluding the treated vessel.

Non-limiting examples of cerebrovascular diseases or disorders include stroke, cerebrovascular inflammation, cerebral hemorrhage and vertebral arterial insufficiency.

Non-limiting examples of peripheral vascular diseases or disorders include gangrene, diabetic vasculopathy, ischemic bowel disease, thrombosis, diabetic retinopathy and diabetic nephropathy.

Non-limiting examples of inflammatory glandular diseases or disorders include pancreatic diseases or disorders, Type I diabetes, thyroid diseases or disorders, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Non-limiting examples of inflammatory gastrointestinal diseases or disorders include colitis, ileitis, Crohn's disease, chronic inflammatory intestinal disease, inflammatory bowel syndrome, chronic inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Non-limiting examples of inflammatory cutaneous diseases or disorders include acne, an autoimmune bullous skin disease, pemphigus vulgaris, bullous pemphigoid, pemphigus foliaceus, contact dermatitis and drug eruption.

Non-limiting examples of inflammatory hepatic diseases or disorders include autoimmune hepatitis, hepatic cirrhosis, and biliary cirrhosis.

Non-limiting examples of inflammatory neurological diseases or disorders include multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), multiple sclerosis, stroke, an inflammatory retinal disease or disorder, an inflammatory ocular disease or disorder, optic neuritis, spongiform encephalopathy, migraine, headache, cluster headache, and stiff-man syndrome.

Non-limiting examples of inflammatory connective tissue diseases or disorders include autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease or disorder, and an autoimmune disease or disorder of the inner ear.

Non-limiting examples of inflammatory renal diseases or disorders include autoimmune interstitial nephritis and/or renal cancer.

Non-limiting examples of inflammatory reproductive diseases or disorders include repeated fetal loss, ovarian cyst, or a menstruation associated disease or disorder.

Non-limiting examples of inflammatory systemic diseases or disorders include systemic lupus erythematosus, systemic sclerosis, septic shock, toxic shock syndrome, and cachexia.

Non-limiting examples of infectious disease or disorder include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome.

Non-limiting examples of inflammatory transplantation-related diseases or disorders include graft rejection, chronic graft rejection, subacute graft rejection, acute graft rejection hyperacute graft rejection, and graft versus host disease or disorder.

Exemplary implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Non-limiting examples of inflammatory tumors include a malignant tumor, a benign tumor, a solid tumor, a metastatic tumor and a non-solid tumor.

Non-limiting examples of inflammatory pulmonary diseases or disorders include asthma, allergic asthma, emphysema, chronic obstructive pulmonary disease or disorder, sarcoidosis and bronchitis.

An examples of a proliferative disease or disorder is cancer.

According to some embodiments of the present invention, the inflammatory disease or disorder is an autoimmune disease or disorder.

Autoimmune diseases or disorders include diseases caused by an immune response such as an autoantibody or cell-mediated immunity to an autoantigen and the like. Representative examples include, but are not limited to, chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

In some embodiments of the invention, the PAF analogs described herein are used in the treatment of multiple sclerosis and arthritis (e.g., chronic rheumatoid arthritis and juvenile rheumatoid arthritis).

In other embodiments of the invention, the PAF analogs described herein are used in the treatment of inflammatory bowel disease, atherosclerosis and psoriasis.

The method described herein may optionally further comprise administering to the subject a therapeutically effective amount of at least one additional compound capable of treating or preventing the inflammatory disease or disorder or a disease or disorder exacerbated thereby.

Exemplary compounds suitable for treating an inflammatory disease or disorder in conjunction with administration of a PAF-analog include, without limitation, a HMGCoA reductase inhibitor (a statin), a mucosal adjuvant, a corticosteroid, a steroidal anti-inflammatory drug, a non-steroidal anti-inflammatory drug, an analgesic, a growth factor, a toxin, a HSP, a beta-2-glycoprotein I, a cholesteryl ester transfer protein (CETP) inhibitor, a peroxisome proliferative activated receptor (PPAR) agonist, an anti-atherosclerosis drug, an anti-proliferative agent, ezetimide, nicotinic acid, an ApoE Milano, and any derivative and analog thereof.

In some embodiments, the additional compound is a steroidal anti-inflammatory drug, a non-steroidal anti-inflammatory drug, or any other drug that is useful in the treatment of the indicated inflammatory disease or disorder to be treated.

Thus, for example, when used in the treatment of multiple sclerosis, an additional active agent that is capable of ameliorating this disease can be utilized in combination with the PAF analogs presented herein.

Similarly, when used in the treatment of arthritis, as described herein, an additional active agent that is capable of ameliorating this disease can be utilized in combination with the PAF analogs presented herein.

The additional compound can be administered concomitant with, prior to or subsequent to administration of the PAF analog, and can alternatively be co-formulated with the PAF analog, as detailed hereinbelow.

Administration of the PAF analogs presented herein can be effected via oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular routes.

As demonstrated in the Examples section that follows, PAF analogs may be effectively administered via an oral route, which enables a safe, easy and convenient administration.

Alternatively, the PAF analogs may be administered by a transmucosal (e.g., transnasal) route, which also allows for safe, easy and convenient administration (e.g., as a nasal spray).

As is detailed herein, the PAF-analogs described herein exert a highly beneficial anti-inflammatory activity and therefore can be utilized in various therapeutic applications. Utilizing these compounds in therapeutic application involves administration thereof either per se, or as a part of a pharmaceutical composition where it is mixed with suitable carriers or excipients.

Thus, according to another aspect of the present invention, there is provided a pharmaceutical composition, which comprises, as an active ingredient, any of the compounds described hereinabove in general Formula I and the accompanying description, and a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the compounds (e.g., CI-302, CI-303 and other compounds depicted in the general Formula I hereinabove) accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

In one embodiment of the invention, the pharmaceutical composition is formulated for mucosal administration.

In one embodiment of the invention, the pharmaceutical compositions are formulated for oral administration.

Alternatively, in other embodiments, the pharmaceutical compositions are designed for nasal, or intraperitoneal administration, as is detailed hereinafter.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin, for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of an inflammatory disease or disorder (e.g., multiple sclerosis, arthritis) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See, e.g., Fingl, et al., 1975, in “The Pharmacological Basis of Therapeutics”, Ch. 1 p. 1).

Dosage amount and interval may be adjusted individually to provide plasma or brain levels of the active ingredient are sufficient to induce or suppress an inflammation (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

Thus, in an embodiment of the present invention, the pharmaceutical composition is packaged in a packaging material and identified in print, on or in the packaging material, for use in the treatment or prevention of an inflammatory disease or disorder, as described herein.

As is further described in detail herein, the pharmaceutical composition can further include an additional compound, which is useful in the treatment or prevention of the inflammatory disease or disorder, as described herein.

The terms “comprises”, “comprising”, “includes”, “including”, “having” and their conjugates mean “including but not limited to”.

The term “consisting of means “including and limited to”.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Experimental Methods

Mice: Female 8-10 week old C67BL/6 mice were purchased from Harlan Laboratories (Israel).

Phospholipids: 1-hexadecyl-2-acetoyl-sn-glycero-3-phosphocholine (PAF), 1-hexadecyl-2-hydroxy-sn-glycero-3-phosphocholine (lyso-PAF; CI-303) and L-a-phosphatidylcholine (PC) were purchased from Sigma-Aldrich (Rehovot, Israel). 1-hexadecyl-2-butyroyl-sn-glycero-3-phosphocholine (CI-302) was purchased from Avanti Polar Lipids (Alabaster, Ala.). Unless indicated otherwise, phospholipids were dissolved in 100% ethanol and further dissolved in PBS to a final concentration of 1% ethanol in PBS for in vitro studies or 0.5% ethanol in PBS for in vivo studies.

Generation of bone-marrow derived dendritic cells (BMDC's): Bone-marrow was flushed out with cold RPMI-1640 medium from mice femur and tibia. A cell suspension was prepared and erythrocytes were removed using red blood cell (RBC) lysis buffer (Beit Haemek, Israel). Cells were washed in phosphate buffer saline (PBS) (Beit Haemek, Israel), and incubated at 4° C. for 15 minutes in buffer containing PBS and 0.5% bovine serum albumin (BSA) with mouse B220 and CD90 microbeads (Miltenyi biotech, Bergisch Gladbach, Germany). Cells were then washed, resuspended in the same buffer, and depleted from B and T cells on a Midi-Macs separation unit through a LD or LS column (Miltenyi Biotech). The depleted cells were then counted, washed and seeded (10⁶/ml) in medium containing RPMI-1640 medium, L-glutamine, β-mercaptoethanol, 10% fetal calf serum (FCS), antibiotics (penicillin, streptomycin) and 20 ng/ml of mouse granulocyte-macrophage colony-stimulating factor (GM-CSF) (Peprotech, Rehovot, Israel). Medium was replaced every other day and cells were used for subsequent experiments on days 5-6 post culturing.

Platelet aggregation assay: Platelet-rich plasma was prepared from citrated whole human blood by centrifugation (200 g for 20 minutes). The final platelet concentration was adjusted to 2×10⁸ platelets/ml with autologous plasma. CI-302 and PAF dissolved in PBS were added to induce platelet activation while the platelet sample was stirred at 1,000 rpm at 37° C. Aggregation was recorded for 5 minutes using an AggRAM 4-channel aggregometer (AggRAM aggregation Remote Analyzer Module; Helena Laboratories).

Cytokine analysis: For the detection of cytokine production, supernatants from 24-hour post-treatment BMDCs were tested with Duoset ELISA kits for mouse IL12/23 p40 (R&D systems, Minneapolis, Minn.).

Preparation of RNA, cDNA and Quantitative Real Time PCR (Q-PCR): RNA was prepared from cells using an RNeasy mini kit (Qiagen, Valencia, Calif.). For cDNA preparation, 1 μg of RNA was combined with oligo-dT for 10 minutes at 70° C., and 1^{St} strand buffer, dithiothreitol (DTT), deoxynucleotides (dNTP) and super-script reverse transcriptase (SS-II) (Invitrogen, Carlsbad, Calif.) were then added for 50 minutes at 42° C. The reaction was ended by incubation for an additional 15 minutes at 70° C. All real time quantitative PCR (q-PCR) reactions were performed using LightCycler Taqman master (Roche Diagnostics, Mannheim, Germany) and the LightCycler (Roche). For tissue mRNA expression, total RNA was extracted from spinal cord using the RNeasy lipid tissue mini kit RNA. Ready sets of probe with primers for q-PCR were purchased from Applied Biosystems (Foster City, Calif.).

Cell stimulation: Mouse BMDCs were pre-incubated for 1 hour with CI-302 at the indicated concentrations before being activated with 10 μg/ml peptidoglycan (InvivoGen, San Diego, Calif.), and supernatants were collected 24 hours later. For RNA preparation, BMDCs were enriched for CD11c+ dendritic cells (>90%) with mouse CD11c microbeads from 5-6 day old cultures over LS columns (Miltenyi Biotech). CD11c+ cells were pre-incubated for 1 hour with 20 μg/ml of CI-302, CI-303 or PC, and then activated for the indicated times with peptidoglycan before collection.

EAE (experimental autoimmune encephalomyelitis) induction: Mice were immunized subcutaneously with an emulsion containing 1.5 mg/ml MOG peptide 35-55 (MEVGWYRSPFSRVVHLYRNGK) (Sigma-Aldrich, Rehovot, Israel) and 2.5 mg/ml complete Freund's adjuvant (CFA), 100 μl in each flank. Pertussis toxin (500 ng in 500 μl PBS) was administered intra-peritoneally immediately and 48 hours after immunization. For prevention of EAE, CI-302 or PBS was given orally once a day for 10 consecutive days beginning 5 days before immunization.

The severity of EAE was assessed utilizing clinical scores determined as follows:

0=normal, 1=limp tail, 2=weakness of hind limbs, 3=hind leg paralysis, 4=hind and fore leg paralysis, 5=moribund or death

Collagen-induced arthritis (CIA) induction: DBA/1 male mice were immunized to induce arthritis by a collagen injection containing complete Freund's adjuvant (CFA) in the base of the tail (day 0) and a booster shot in the flank (day 21). Mice were followed for arthritis development until day 39. Administration by gavage of the tested compounds and control substances began on day 22 and was carried out on a daily basis (6 times per week).

The severity of arthritis was assessed as follows:

Each paw is scored on a scale of 0-4 for the degree of swelling, erythema, and deformity of the joints (maximum score 16 per animal) as follows [Brackertz et al., 1977]:

0=normal
1=slight erythema and/or swelling of the ankle or wrist or toes
2=moderate erythema and/or swelling of the ankle or wrist
3=severe erythema and/or swelling of the ankle or wrist (or moderate in combination of all toes)
4=complete erythema and swelling of toes or fingers and ankle or wrist, and inability to bend the ankle or wrist

Western blot: Thioglycolate-stimulated peritoneal macrophages were treated for 10 minutes with 20 μg/ml of CI-302. 20 μg/ml of phosphatidylcholine or solvent served as negative controls, whereas CI-201 served as a positive control. Whole cell lysates were resolved by SDS-PAGE on 10% Tris/HCl denaturing gel, transferred to nitrocellulose membranes and blotted with anti-phosphotyrosine antibody. Blotting with anti-ERK1/2 antibody served as a loading control.

### Results

CI-302 does not Induce Platelet Aggregation:

The conversion of PAF to its lyso form (CI-303) by acetylhydrolase renders it biologically inactive [Blank et al., 1981]. However, other PAF derivatives may still possess the potential to induce platelet aggregation [Baumgartner and Hadvary, 1983]. Consequently, CI-302 was tested for PAF-like activity on human platelets. The structures of PAF, CI-303 and CI-302 are shown in FIG. 1.

CI-302 was applied to human platelets for 5 minutes in escalating concentrations of up to 1 mM, and platelet aggregation was measured. As shown in FIGS. 2-4, no platelet aggregation was observed with CI-302, whereas treatment with PAF resulted in a substantial platelet aggregation.

CI-302 and CI-303 Inhibit IL-12/23 p40 Production from Activated BMDCs:

Oxidized phospholipids have been found to inhibit the production of cytokines by dendritic cells [Bluml et al., 2005]. To test whether CI-302 could display a similar regulatory effect, BMDCs were activated with peptidoglycan in the presence of escalating CI-302 concentrations and supernatants were collected 24 hours later and tested for IL-12/23 p40 production by ELISA.

As shown in FIG. 5A, CI-302 significantly inhibited production of p40 by activated BMDCs in a dose-dependent manner, with a maximal reduction of 45% attained with the highest CI-302 concentration tested.

As shown in FIG. 5B, CI-303 also inhibited production of p40, with a statistically significant reduction obtained with 20 and 5 μg/ml CI-303.

We next attempted to determine the mechanism by which CI-302 and CI-303 inhibited p40 production. Therefore, q-PCR for p40 was performed on RNA samples generated from BMDCs activated with peptidoglycan in the presence of CI-302 or CI-303.

As shown in FIG. 6, both PAF analogs inhibited p40 mRNA expression considerably at all time points tested, whereas PC showed little or no effect. These results suggest that CI-302 and CI-303 down-regulate p40 expression by BMDCs at the transcription level.

CI-302 Prevents MOG Induced EAE:

The central role played by p40 in the development of EAE has been substantiated through several studies. Mice targeted in their p40 gene were protected from EAE induction and treatment with anti p40 antibodies prevented EAE development [Becher et al., 2002; Gran et al., 2002; Segal et al., 1998; Brok et al., 2002]. Based on these studies and the results described above, it is hypothesized that in vivo treatment with CI-302 could prevent induction of EAE. To test the hypothesis, mice were orally administered with CI-302 five days before and after immunization with MOG peptide 35-55 and monitored for clinical signs.

As shown in FIG. 7, CI-302 at a dose of 0.4 mg/kg resulted in close to complete protection against EAE development, while administration of 0.04 mg/kg CI-302 resulted in a noticeable though milder protection against disease induction.

In order to evaluate the effect of CI-302 administration on inflammation in the target tissue, q-PCR for pro-inflammatory chemokines and cytokines was carried out on spinal cords taken from PBS and CI-302 (0.4 mg/kg) treated groups.

As shown in FIGS. 8A-F, the mRNA expression level of all tested genes from CI-302 treated mice was reduced relative to the PBS-treated control group. Specifically, p40 and p35 gene transcription by antigen-presenting cells and CCL2 (MCP-1) transcription were reduced in CI-302 treated mice, (FIGS. 8A-C) along with transcription of the Tₕ1 cytokines IFN-γ and TNF-α (FIGS. 8D-E). In addition, CD4 mRNA levels were considerably lower in CI-302 treated mice than in control mice (FIG. 8F), suggesting a reduced presence of infiltrating T-helper cells in the target tissue. Taken together, these results suggest that CI-302 inhibited disease induction by restricting the infiltration of pathogenic CD4+ T-cells into the target tissue.

CI-302 Inhibits Development of Collagen-Induced Arthritis (CIA):

Anti-inflammatory compounds may have a beneficial effect in treatment of arthritis. Hence, the effect of PAF-analogs in a mouse CIA model was investigated.

As shown in FIG. 9, treatment with 0.4 mg/kg of CI-302 in a CIA mouse model decreased arthritis severity throughout the study period.

CI-302 Inhibits Tyrosine Phosphorylation in Macrophages:

The effect of PAF-analogs on cell signaling in immune cells was studied by examination of tyrosine phosphorylation in peritoneal macrophages.

As shown in FIG. 10, CI-302 strongly reduced the level of phosphotyrosine observed in peritoneal macrophages by Western blotting, indicating either significant inhibition of tyrosine phosphorylation or induction of de-phosphorylation events.

### LIST OF REFERENCES CITED

Bailey S L, Schreiner B, McMahon E J, Miller S D. CNS myeloid DCs presenting endogenous myelin peptides ‘preferentially’ polarize CD4+ T(H)-17 cells in relapsing EAE. Nat Immunol (2007) 8:172-180.
Baumgartner H R and Hadvary P. Activation of human and rabbit blood platelets by synthetic structural analogs of platelet activating factor. Thrombosis Research (1983) 30:143-156.
Becher B, Prat A, Antel J P. Brain-immune connection: immuno-regulatory properties of CNS-resident cells. Glia (2000) 29:293-304.
Becher B, Durell B G, Noelle R J. Experimental autoimmune encephalitis and inflammation in the absence of interleukin-12. J Clin Invest (2002) 110:493-497.
Blank M L, Lee T, Fitzgerald V, Snyder F. A specific acetylhydrolase for 1-alkyl-2-acetyl-sn-glycero-3-phosphocholine (a hypotensive and platelet-activating lipid). J Biol Chem (1981) 256:175-178.
Bluml S, Kirchberger S, Bochkov V N, Kronke G, Stuhlmeier K, Majdic O, Zlabinger G J, Knapp W, Binder B R, Stockl J, Leitinger N. Oxidized phospholipids negatively regulate dendritic cell maturation induced by TLRs and CD40. J Immunol (2005) 175:501-508.
Bochkov V N, Kadl A, Huber J, Gruber F, Binder B R, Leitinger N. Protective role of phospholipid oxidation products in endotoxin-induced tissue damage. Nature (2002) 419:77-81.
Brackertz D, Mitchell G F, Mackay I R. Antigen-induced arthritis in mice. I. Induction of arthritis in various strains of mice. Arthritis and Rheumatism (1977) 20:841-850.
Brok H P, van Meurs M, Blezer E, Schantz A, Peritt D, Treacy G, Laman J D, Bauer J, 't Hart B A. Prevention of experimental autoimmune encephalomyelitis in common marmosets using an anti-IL-12p40 monoclonal antibody. J Immunol (2002) 169:6554-6563.
Deshpande P, King I L, Segal B M. Cutting edge: CNS CD11c+ cells from mice with encephalomyelitis polarize Th17 cells and support CD25+CD4+ T cell-mediated immunosuppression, suggesting dual roles in the disease process. J Immunol (2007) 178:6695-6699.
Fierz W, Endler B, Reske K, Wekerle H, Fontana A. Astrocytes as antigen-presenting cells. I. Induction of 1a antigen expression on astrocytes by T cells via immune interferon and its effect on antigen presentation. J Immunol (1985) 134:3785-3793.
Ford A L, Foulcher E, Lemckert F A, Sedgwick J D. Microglia induce CD4 T lymphocyte final effector function and death. J Exp Med (1996) 184:1737-1745.
Gran B, Zhang G X, Yu S, Li J, Chen X H, Ventura E S, Kamoun M, Rostami A. IL-12p35-deficient mice are susceptible to experimental autoimmune encephalomyelitis: evidence for redundancy in the IL-12 system in the induction of central nervous system autoimmune demyelination. J Immunol (2002) 169:7104-7110.
Greter M, Heppner F L, Lemos M P, Odermatt B M, Goebels N, Laufer T, Noelle R J, Becher B. Dendritic cells permit immune invasion of the CNS in an animal model of multiple sclerosis. Nat Med (2005) 11:328-334.
Heery J M, Kozak M, Stafforini D M, Jones D A, Zimmerman G A, McIntyre T M, Prescott S M. Oxidatively modified LDL contains phospholipids with platelet-activating factor-like activity and stimulates the growth of smooth muscle cells. J Clin Invest (1995) 96:2322-2330.
Lehr H A, Weyrich A S, Saetzler R K, Jurek A, Arfors K E, Zimmerman G A, Prescott S M, McIntyre T M. Vitamin C blocks inflammatory platelet-activating factor mimetics created by cigarette smoking. J Clin Invest (1997) 99:2358-2364.
Marathe G K, Davies S S, Harrison K A, Silva A R, Murphy R C, Castro-Faria-Neto H, Prescott S M, Zimmerman G A, McIntyre T M. Inflammatory platelet-activating factor-like phospholipids in oxidized low density lipoproteins are fragmented alkyl phosphatidylcholines. J Biol Chem (1999) 274:28395-28404.
Marathe G K, Zimmerman G A, Prescott S M, McIntyre T M. Activation of vascular cells by PAF-like lipids in oxidized LDL. Vascul Pharmacol (2002) 38:193-200.
McMahon E J, Bailey S L, Castenada C V, Waldner H, Miller S D. Epitope spreading initiates in the CNS in two mouse models of multiple sclerosis. Nat Med (2005) 11:335-339.
Segal B M, Dwyer B K, Shevach E M. An interleukin (IL)-10/IL-12 immunoregulatory circuit controls susceptibility to autoimmune disease. J Exp Med (1998) 187:537-546.
Slavin A J, Soos J M, Stuve O, Patarroyo J C, Weiner H L, Fontana A, Bikoff E K, Zamvil S S. Requirement for endocytic antigen processing and influence of invariant chain and H-2M deficiencies in CNS autoimmunity. J Clin Invest (2001) 108:1133-1139.
Smiley P L, Stremler K E, Prescott S M, Zimmerman G A, McIntyre T M. Oxidatively fragmented phosphatidylcholines activate human neutrophils through the receptor for platelet-activating factor. J Biol Chem (1991) 266:11104-11110.
Tokumura A, Toujima M, Yoshioka Y, Fukuzawa K. Lipid peroxidation in low density lipoproteins from human plasma and egg yolk promotes accumulation of 1-acyl analogues of platelet-activating factor-like lipids. Lipids (1996) 31:1251-1258.
Walton K A, Cole A L, Yeh M, Subbanagounder G, Krutzik S R, Modlin R L et al. Specific phospholipid oxidation products inhibit ligand activation of toll-like receptors 4 and 2. Arterioscler Thromb Vasc Biol (2003) 23:1197-1203.

1-16. (canceled)

17. A method of treating an inflammatory disease or disorder in a subject in need thereof, the method comprising administering the subject a therapeutically effective amount of at least one PAF-analog of general Formula I: 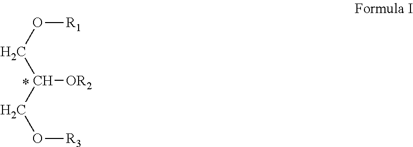
wherein:
* denotes a chiral or non-chiral carbon atom, having a S-configuration and/or a R-configuration;
R₁ is an alkyl chain 16 to 18 carbon atoms in length;
R₂ is selected from the group consisting of hydrogen and an acyl group having the formula:
—C(═O)—(CH₂)n-H
wherein n equals 0, 2, 3, 4, 5, 6 or 7; and
R₃ is selected from the group consisting of phosphate, phosphocholine, phosphoethanolamine, phosphoserine, phosphoinositol, alkyl, aryl, cycloalkyl, carboxy, saccharide, ethylphosphocholine, phosphorylmethanol, phosphorylethanol, phosphorylpropanol, phosphorylbutanol, phosphoethanolamine-N-lactose, phosphoethanolamine-N-[methoxy(propylene glycol)], phosphoinositol-4-phosphate, phosphoinositol-4,5-biposphonate, pyrophosphate, phosphoethanolamine-diethylenetriamine-pentaacetate, dinitrophenyl-phosphoethanolamine and phosphoglycerol,
or a pharmaceutically acceptable salt thereof,
thereby treating the inflammatory disease or disorder.

18. The method of claim 17, wherein R₃ is phosphocholine.

19. The method of claim 17, wherein R₁ is hexadecyl.

20. The method of claim 17, wherein R₂ is hydrogen.

21. The method of claim 17, wherein n equals 3.

22. The method of claim 18, wherein R₁ is hexadecyl.

23. The method of claim 22, wherein R₂ is hydrogen.

24. The method of claim 23, wherein n equals 3.

25. The method of claim 17, wherein said PAF-analog is selected from the group consisting of 1-hexadecyl-2-butyroyl-sn-glycero-3-phosphocholine, 1-octadecyl-2-butyroyl-sn-glycero-3-phosphocholine, 1-hexadecyl-2-hydroxy-sn-glycero-3-phosphocholine and 1-octadecyl-2-hydroxy-sn-glycero-3-phosphocholine.

26. The method of claim 17, wherein said inflammatory disease or disorder is selected from the group consisting of an idiopathic inflammatory disease or disorder, a chronic inflammatory disease or disorder, an acute inflammatory disease or disorder, an autoimmune disease or disorder, an infectious disease or disorder, an inflammatory malignant disease or disorder, an inflammatory transplantation-related disease or disorder, an inflammatory degenerative disease or disorder, a disease or disorder associated with a hypersensitivity, an inflammatory cardiovascular disease or disorder, an inflammatory cerebrovascular disease or disorder, a peripheral vascular disease or disorder, an inflammatory glandular disease or disorder, an inflammatory gastrointestinal disease or disorder, an inflammatory cutaneous disease or disorder, an inflammatory hepatic disease or disorder, an inflammatory neurological disease or disorder, an inflammatory musculo-skeletal disease or disorder, an inflammatory renal disease or disorder, an inflammatory reproductive disease or disorder, an inflammatory systemic disease or disorder, an inflammatory connective tissue disease or disorder, an inflammatory tumor, necrosis, an inflammatory implant-related disease or disorder, an inflammatory aging process, an immunodeficiency disease or disorder and an inflammatory pulmonary disease or disorder.

27. The method of claim 17, wherein said inflammatory disease or disorder is an autoimmune disease or disorder.

28. The method of claim 27, wherein said autoimmune disease or disorder is selected from the group consisting of chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

29. The method of claim 28, wherein said autoimmune disease or disorder is selected from the group consisting of inflammatory bowel disease, atherosclerosis, psoriasis, chronic rheumatoid arthritis, juvenile rheumatoid arthritis and multiple sclerosis.

30. The method of claim 17, further comprising administering to the subject a therapeutically effective amount of at least one additional compound capable of treating or preventing said inflammatory disease or disorder.

31. The method of claim 30, wherein said at least one additional compound is selected from the group consisting of a HMGCoA reductase inhibitor (a statin), a mucosal adjuvant, a corticosteroid, a steroidal anti-inflammatory drug, a non-steroidal anti-inflammatory drug, an analgesic, a growth factor, a toxin, a HSP, a beta-2-glycoprotein I, a cholesteryl ester transfer protein (CETP) inhibitor, a peroxisome proliferative activated receptor (PPAR) agonist, an anti-atherosclerosis drug, an anti-proliferative agent, ezetimide, nicotinic acid, an ApoE Milano, and any derivative and analog thereof.

32. A pharmaceutical composition comprising a PAF-analog of general Formula I: 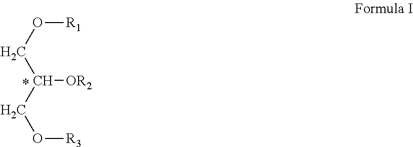
wherein:
* denotes a chiral or non-chiral carbon atom, having a S-configuration and/or a R-configuration;
R₁ is an alkyl chain 16 to 18 carbon atoms in length;
R₂ is selected from the group consisting of hydrogen and an acyl group having the formula:
—C(═O)—(CH₂)n-H
wherein n equals 0, 2, 3, 4, 5, 6 or 7; and
R₃ is selected from the group consisting of phosphate, phosphocholine, phosphoethanolamine, phosphoserine, phosphoinositol, alkyl, aryl, cycloalkyl, carboxy, saccharide, ethylphosphocholine, phosphorylmethanol, phosphorylethanol, phosphorylpropanol, phosphorylbutanol, phosphoethanolamine-N-lactose, phosphoethanolamine-N-[methoxy(propylene glycol)], phosphoinositol-4-phosphate, phosphoinositol-4,5-biposphonate, pyrophosphate, phosphoethanolamine-diethylenetriamine-pentaacetate, dinitrophenyl-phosphoethanolamine and phosphoglycerol,
or a pharmaceutically acceptable salt thereof,
and a pharmaceutical acceptable carrier, the composition being packaged in a packaging material and identified in print, in or on the packaging material for use in the treatment of an inflammatory disease or disorder.

33. The composition of claim 32, wherein R₃ is phosphocholine.

34. The composition of claim 33, wherein R₁ is hexadecyl.

35. The composition of claim 34, wherein R₂ is hydrogen.

36. The composition of claim 34, wherein n equals 3.

37. The composition of claim 32, wherein said PAF-analog is selected from the group consisting of 1-hexadecyl-2-butyroyl-sn-glycero-3-phosphocholine, 1-octadecyl-2-butyroyl-sn-glycero-3-phosphocholine, 1-hexadecyl-2-hydroxy-sn-glycero-3-phosphocholine and 1-octadecyl-2-hydroxy-sn-glycero-3-phosphocholine.

38. The composition of claim 32, wherein said inflammatory disease or disorder is selected from the group consisting of an idiopathic inflammatory disease or disorder, a chronic inflammatory disease or disorder, an acute inflammatory disease or disorder, an autoimmune disease or disorder, an infectious disease or disorder, an inflammatory malignant disease or disorder, an inflammatory transplantation-related disease or disorder, an inflammatory degenerative disease or disorder, a disease or disorder associated with a hypersensitivity, an inflammatory cardiovascular disease or disorder, an inflammatory cerebrovascular disease or disorder, a peripheral vascular disease or disorder, an inflammatory glandular disease or disorder, an inflammatory gastrointestinal disease or disorder, an inflammatory cutaneous disease or disorder, an inflammatory hepatic disease or disorder, an inflammatory neurological disease or disorder, an inflammatory musculo-skeletal disease or disorder, an inflammatory renal disease or disorder, an inflammatory reproductive disease or disorder, an inflammatory systemic disease or disorder, an inflammatory connective tissue disease or disorder, an inflammatory tumor, necrosis, an inflammatory implant-related disease or disorder, an inflammatory aging process, an immunodeficiency disease or disorder and an inflammatory pulmonary disease or disorder.

39. The pharmaceutical composition of claim 32, further comprising a therapeutically effective amount of at least one additional compound capable of treating or preventing said inflammatory disease or disorder.

40. The composition of claim 39, wherein said at least one additional compound is selected from the group consisting of a HMGCoA reductase inhibitor (a statin), a mucosal adjuvant, a corticosteroid, a steroidal anti-inflammatory drug, a non-steroidal anti-inflammatory drug, an analgesic, a growth factor, a toxin, a HSP, a beta-2-glycoprotein I, a cholesteryl ester transfer protein (CETP) inhibitor, a peroxisome proliferative activated receptor (PPAR) agonist, an anti-atherosclerosis drug, an anti-proliferative agent, ezetimide, nicotinic acid, an ApoE Milano, and any derivative and analog thereof.
